(19) United States
(12) Patent Application Publication
(10) Pub. No.: US 2007/0161864 A1
(43) Pub. Date: Jul. 12, 2007

(54) Laparoscopic Tissue Retractor

(76) Inventor: Dale A. Sloan, Fort Wayne, IN (US)

Correspondence Address: FROST BROWN TODD, LLC, 2200 PNC CENTER, 201 E. FIFTH STREET, CINCINNATI, OH, 45202, US

(21) Appl. No.: 11/621,243

(22) Filed: Jan. 9, 2007

Related U.S. Application Data
(60) Provisional application No. 60/757,237, filed on Jan. 9, 2006.

(51) Int. Cl. A61B 1/32

(52) U.S. Cl. 600/204

(57) ABSTRACT

The device is a tissue retractor including versions having a combination of an anchor and a tissue retention member for the displacement of tissue obstacles and the like during medical procedures.

### PRIORITY

The application claims priority from the disclosure of U.S. Provisional Patent Application Ser. No. 60/757,237 entitled Laparoscopic Tissue Retractor, filed Jan. 9, 2006, which is herein incorporated by reference in its entirety.

### BACKGROUND

The present disclosure relates to devices, systems and methods used for retracting organs and/or body tissue during surgical procedures and, more particularly, to endoscopic or laparoscopic apparatus, systems and methods for retracting or positioning body tissue and/or body organs during minimally invasive surgery.

As a result of the recent technological improvements in surgical instruments, surgical procedures, using minimally invasive techniques, such as endoscopic and laparoscopic, are routinely performed, where such surgical procedures generally cause less trauma to the patient.

In endoscopic and laparoscopic surgical procedures, tissue and organs located in the area of operation frequently obstruct a surgeon's view or become obstacles in the workspace. Generally, laparoscopic surgical procedures involve the introduction of a gas, such as, carbon dioxide, to insufflate a body cavity such as, for example, the abdomen, to provide a working area for the surgeon. After the abdomen is insufflated, a trocar device is commonly used to puncture the peritoneum to provide an access port by way of a cannula through the abdominal wall for the introduction of surgical instrumentation. Generally, a trocar or cannula is placed through the abdominal wall for each piece of surgical instrumentation which is necessary to carry out the surgical procedure. In this manner, the surgeon may view the surgical site through an endoscope provided through a first trocar/cannula, and utilize a second trocar/cannula to introduce a surgical instruments. However, the surgeon must also frequently navigate the instruments around tissue and/or organs within the patient.

Although the insufflation gas expands the abdomen to permit the surgeon to view the surgical site, it is often necessary to manipulate the internal organs or tissues to provide a clear path to the surgical objective. In the past, instruments have been utilized that pull on the organs or tissues to move them out of the way to provide a clear visual path for the surgeon. Such devices are disclosed in, for example, U.S. Pat. No. 4,654,028 to Suma, U.S. Pat. No. 4,909,789 to Taguchi et al., and U.S. Pat. No. 5,195,505 to Josefsen. Other retractor devices include collapsible fingers and are disclosed in, for example, U.S. Pat. No. 5,456,695 to Herve Dallemange, U.S. Pat. No. 4,190,042 to Sinnreich, and U.S. Pat. No. 4,744,363 to Hasson. Other devices include retractors having expandable frames for supporting expandable latex sheaths or covers, such as those described in U.S. Pat. No. 5,178,133 to Pena.

It would be advantageous to provide a laparoscopic organ retraction system having sufficient strength and durability to retract body organs from the operative site that combines both a tissue anchor and a tissue retraction member. It would be further advantageous to provide an organ retraction system that is cost effective and easy to use.

### BRIEF DESCRIPTION OF THE FIGURES

In accordance with versions herein, it is believed the present invention will be better understood from the following description taken in conjunction with the accompanying drawings. The drawings and detailed description that follow are intended to be merely illustrative and are not intended to limit the scope of the invention.

FIG. 1 presents a perspective view of an organ retraction instrument shown in the closed position;

FIG. 2 presents a perspective view of an organ retraction instrument shown in the open position;

FIG. 3 presents a perspective view of an alternate version of an organ retraction instrument shown grasping and retracting tissue within a patient;

FIG. 4 presents a perspective view of an alternate version of an organ retraction instrument shown in the open position;

FIG. 5 presents a perspective view of an alternate version of an organ retraction instrument shown in the closed position; and

FIG. 6 presents a perspective view of an alternate version of an organ retraction instrument shown grasping and retracting tissue within a patient.

### DETAILED DESCRIPTION OF THE INVENTION

The present invention shall be discussed in terms of endoscopic and laparoscopic procedures and apparatus. However, use herein of terms such as “endoscopic” and “laparoscopic”, among others, should not be construed to limit the present invention to an apparatus for use only in conjunction with such procedures. To the contrary, it is believed that versions herein may find use in procedures wherein an organ or other tissue may be advantageously displaced such as, for example, in the abdomen and thoracic cavity.

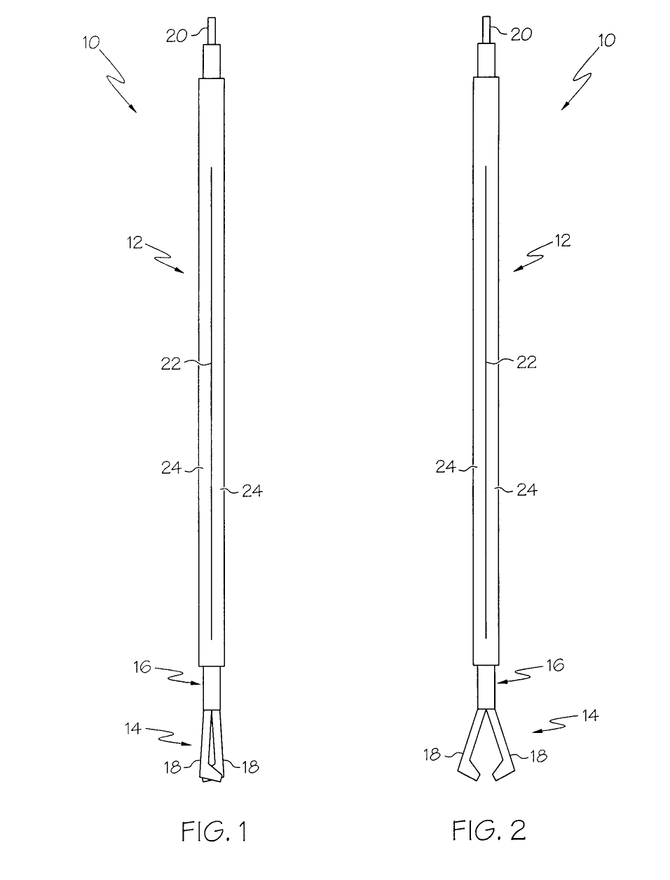

Referring to the figures, FIGS. 1-2 illustrate one version of a tissue or organ retractor 10 adapted for laparoscopic insertion, having a proximal and a distal end, shown in a “closed” position (FIG. 1) and an “open” position (FIG. 2). In one version, the retractor 10 includes a retraction member 12 and an anchor 14, where the retraction member 12 and the anchor 14 are coupled with a support member 16 and cooperate to displace an organ, such as the liver, or other tissue.

The anchor 14 may include a set of grasping jaws 18 operably configured to retain tissue therebetween, such as the patient's diaphragm, or otherwise secure the retractor 10 for deployment. The grasping jaws 18 may articulate in a scissor-like motion and may be articulated by, for example, actuating a drive member 20 attached thereto. The drive member 20 may be, for example, a rod or tube coaxial with the support member 16 operably configured to translate therethrough.

The grasping jaws 18 may be connected to the support member 16 via a hinge (not shown) and to the drive member 20 such that actuating the drive member 20 articulates the grasping jaws 18 about the hinges to an open and closed position. After insertion of the retractor 10 into a patient's body, the grasping jaws may be opened with the drive member 20 and positioned to accept tissue therebetween. The drive member 20 may then be actuated to close the grasping jaws 18 such that the retractor 10 is secured to the tissue.

It will be appreciated that the anchor 14 may include any suitable means for securing the retractor 10 to tissue prior to deployment such as, for example, with suction or with an anchor adapted to puncture and hold tissue. The anchor 14 and/or drive member 20 may be articulated or actuated by any suitable means including, but not limited to, a handle (not shown) having a trigger, screw, electric, and/or mechanical drive system.

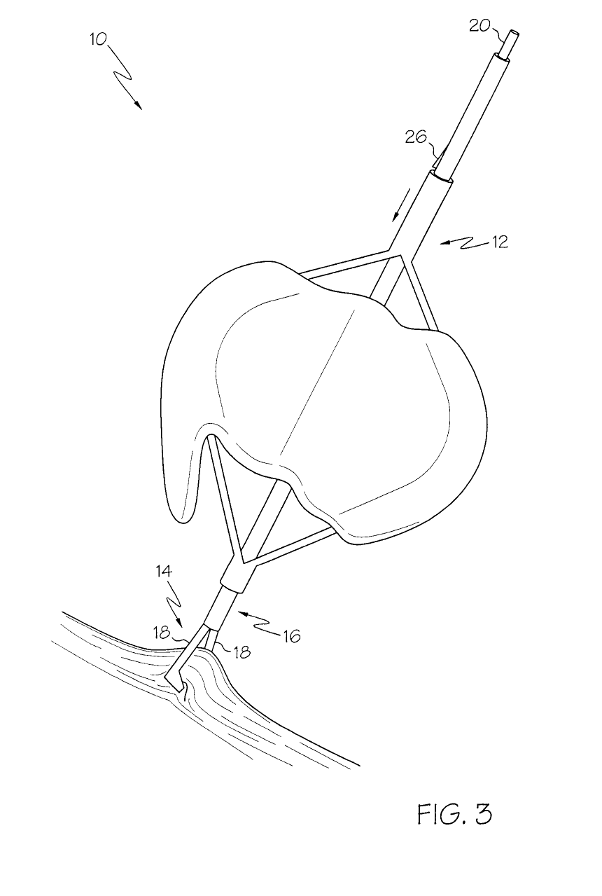

Referring to FIG. 3, once the retractor 10 is secured to tissue, the retraction member 12 may be deployed. In one version, the retraction member 12 is an annular sleeve having a proximal end and a distal end. The distal end of the retraction member 12 may be permanently coupled at about the distal end of the support member 16 while the proximal end of the retraction member 12 may be free to translate longitudinally. Referring to FIG. 2, the retraction member 12 may include longitudinal slits defining supports 24 that expand (FIG. 3) on, for example, a living hinge when the proximal end of the retraction member 12 is pushed distally. In one version, the retraction member 12 and/or supports 24 are configured from a flexible, elastomeric, and/or memory retention material, such as nitinol, and are operably configured to expand or deploy with sufficient strength to move, retain, and/or hold tissue.

It will be appreciated that the retention member 12 and/or supports 24 may be constructed from any suitable material, such as a metal frame or polymeric frame, and may be configured to any suitable shape during and prior to deployment. For example, the retention member may be inflatable in whole or in part, substantially planar or paddle-shaped, and/or may have any other suitably shaped frame. It will be appreciated that the retention member 12 may include rigid, semi-rigid, memory retention, and/or flexible components such as, for example, where the proximal end and the distal end of the retention member are rigid and the supports 24 located therebetween are constructed from a flexible material. It will be further appreciated that the retention member 12 may include one or a plurality of supports 24 where, for example, the retention member 12 has four supports (not shown) expandable around the radius of the retractor 10 in increments of ninety degrees.

In a further version (not shown), the retraction member 12 may be operably configured to rotate about the support member 16 such that the surgeon may position the supports 24 in an advantageous position for retracting tissue while maintaining the anchor 14 at a desired tissue location. Rotation may be accomplished, for example, with a tongue and groove connection between the retention member 12 and the support member 16.

Referring to FIGS. 2-3, in one version, the retractor 10 includes a catch 26 operably configured to retain the shape of the retention member 12 when the supports 24 are deployed. The catch 26 may, for example, be held within a recess (not shown) within the support member 16 and may be retained therein when covered by the retention member 12. When the retention member 12 is actuated distally, the catch 26 may become unsheathed and may assume the position illustrated in FIG. 2. The catch 26 may be opened, for example, automatically by providing the catch 26 with a spring bias that pushes the catch 26 outward when unsheathed. It will also be appreciated that the catch 26 may be manually engaged and/or disengaged by a surgeon via a lever or the like. In an alternate version, the catch 26 may be positionable to expand or retract the supports 24 at a desired location. For example, the catch 26 may ride in a longitudinal track (not shown) and may be actuated proximally and distally, thereby expanding and retracting the supports 24, before being locked into place to secure the supports 24 at a desired position and/or expansion.

It will be appreciated that catch 26, or any other suitable assembly or component for sizing, shaping, and/or securing the retention member 12, are contemplated. For example, an annular collar (not shown), having a lateral set screw therein, may be placed about the support member 16 such that longitudinally translating the collar along the support member 16 opens and closes the retention member 12. When the retention member 12 is set to a desirable shape and/or configuration, the set screw may be tightened to hold the annular collar in place. The annular collar may, for example, be adjusted throughout the procedure or may be left in place until removal is necessary.

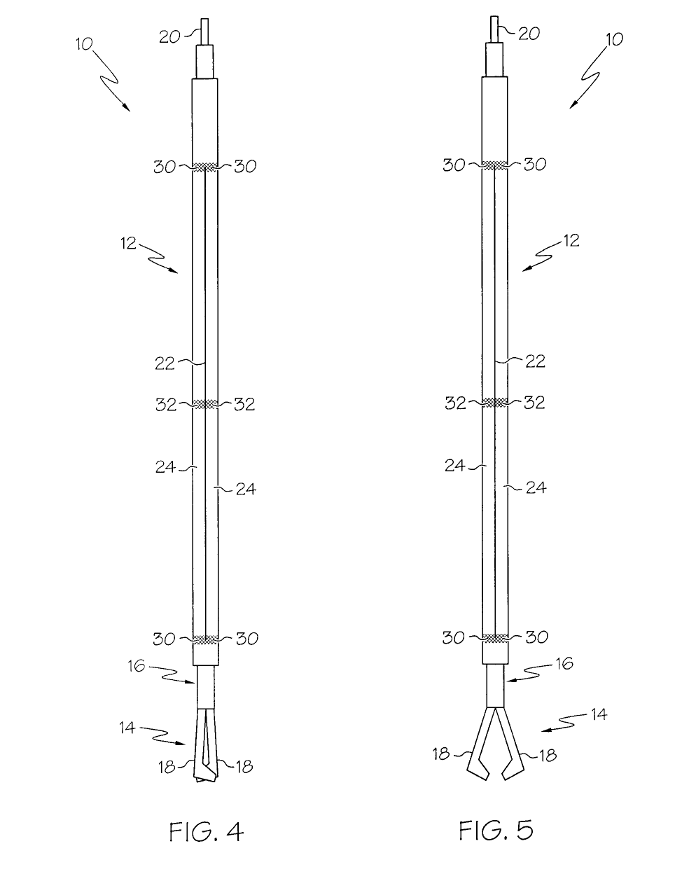

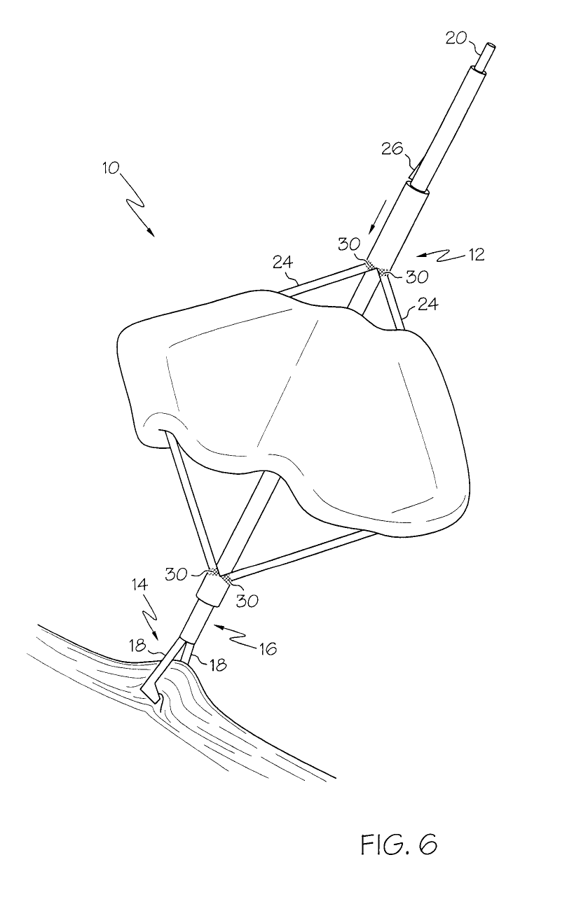

Referring to FIGS. 4-6, an alternate version of the retractor 10 is disclosed having hinges 30 positioned at the intersection of the supports 24 and the retraction member 12. Additionally, an alternate embodiment may include one or a plurality of hinges 32 positioned on the supports 24.

Deploying the retention member 12 of the retractor 10 may provide a surgeon with, for example, more visibility or a better workspace during a laparoscopic procedure by displacing or otherwise moving tissue obstacles or the like. Securing the retractor 10 to the patient internally with the anchor 14 may reduce the number of cumbersome or awkward instruments in the surgical field outside the patient's body. Additionally, by integrating an anchor with a retention member, the surgeon may be provided with an efficient, cost-efficient, and easy to use instrument that is effective without taking up a significant amount of space. By providing flexible or jointed supports, versions of the present invention may provide a broad surface with which to restrain or hold tissue, while still having the benefit of an instrument that can be guided through, for example, a 5 mm or 10 mm port, although it will be appreciated that versions herein may be used independently of such ports.

We claim:

1. A laparoscopic tissue retractor comprising:
(a) a retraction member,
(b) an anchor coupled with the retraction member, and
(c) a support member associated with the retraction member and the anchor.
